(19) United States
(12) Patent Application Publication
(10) Pub. No.: US 2008/0200533 A1
(43) Pub. Date: Aug. 21, 2008

(54) Drug or Pharmaceutical Compounds and a Preparation Thereof

(76) Inventor: Ramu Krishnan, Chennai (IN)

Correspondence Address: BROOKS KUSHMAN P.C., 1000 TOWN CENTER, TWENTY-SECOND FLOOR, SOUTHFIELD, MI, 48075, US

(21) Appl. No.: 11/994,752

(22) PCT Filed: Jun. 29, 2006
(86) PCT No.: PCT/IN2006/000222

§ 371 Date: Jan. 4, 2008

(30) Foreign Application Priority Data
Jul. 4, 2005 (IN) 861/CHE/2005

(51) Int. Cl. A61K 31/40; C07D 239/42; C07D 207/34; C07K 2/00; C07D 205/08; C07D 215/44; C07J 9/00

(52) U.S. Cl. 514/423; 544/332; 548/537; 530/300; 540/200; 546/159; 552/544

(57) ABSTRACT

The administration of pharmaceuticals of drugs which are having less solubility, lower bioavailability, lower bioabsorbability, less rate of absorption has become a big challenge in day to day life. Therefore an attempt has been made to prepare a complex modified form of the said pharmaceutical or drug such that the modified complex drugs or pharmaceuticals exhibits the enhanced properties of solubility, bioavailability, bioabsorbability and rate of absorption despite the increased complexity of the molecule. Surprisingly such modification was found to enhance retentivity of the active drug ingredient in the blood. Higher amounts of the active drug ingredient has shown lower toxicity.

The present invention relates to improved pharmaceutical compounds and method of preparation of the same. The present invention aims to modify the relatively insoluble drugs into highly soluble form so that the compounds of the present invention enable better administration of the said drugs. The compounds of the present invention also have improved bioavailability and bioabsorptivity of the important drugs such as described in the present invention.

Most often drugs are compounds foreign to the body. Drug absorption, distribution, and elimination are therefore determinants of the intensity of drug effect.

Drug absorption is determined by physicochemical properties of drugs, their formulations, and routes of administration. Drug products—the actual dosage forms (e.g., tablets, capsules, solutions), consisting of the drug plus other ingredients are formulated to be administered by various routes, including oral, buccal, sublingual, rectal, parenteral, topical, and inhalational.

In general most of the drugs or pharmaceuticals are solid complex of organic compounds. These organic compounds are generally not soluble in water. Hence the active ingredients are mixed with certain adjuvant and formulated into tablets, capsules etc., It is also equally important to have drugs in liquid forms such as syrups for oral administration for the reasons obviously known. Therefore the aim of the present invention is to invent modified molecular structures of the drugs and pharmaceuticals either in the solid form, which are highly soluble in aqueous solutions or non-aqueous solutions or directly in the suitable liquid forms. The present invention emphasizes using organic acids, bases and relevant anion radicals for modifying the drug or pharmaceutical structures into a form wherein it is more bioavailable and bioabsorbable.

Also the present invention emphasizes about the solubility of the drug molecules in fats, oils and other non-aqueous solutions. The aim is that the complex drug molecule should reach the site of action without difficulty and without undergoing any undesired change in the structure.

When given by most routes (excluding intra venous), a drug must traverse several semi permeable cell membranes before reaching the systemic circulation. These membranes are biologic barriers that selectively inhibit the passage of drug molecules and are composed primarily of a bimolecular lipid matrix, containing mostly cholesterol and phospholipids. The lipids provide stability to the membrane and determine its permeability characteristics. Globular proteins of various sizes and composition are embedded in the matrix; they are involved in transport and function as receptors for cellular regulation. Drugs may cross a biological barrier by passive diffusion, facilitated passive diffusion, active transport, or pinocytosis.

The ability of a drug to remain in its soluble form at the point where it is absorbed in the digestive track is one of the most important criteria determining its bioabsorbability. Most drugs precipitate at a pH of between and 8, the pH as is found in the intestine of an organism. Surprisingly our invention results in an oil soluble drugs to remain soluble till the pH of at least up to 9 ensuring best bioabsorption.

Nano-technology generally refers to a process wherein the particle size is reduced to a nano scale (1 billionth of a meter). The effect of the drugs remaining in soluble form in the varied pH's of the digestive track ensures that the drug molecules do not coagulate/precipitate and their size remain in the nano scale ensuring best bioabsorption. Uniqueness of this invention is also that the suitably chemically modified drug permeates the digestive track and reaches the blood stream much faster than that of the conventional drug. Thus drugs, which are required to be given in fasting condition, can be suitably modified ensuring that the time required for fasting can be sizeably reduced. The most interesting point of our invention is the applicability of novelty and inventive concept by the chemical modification of the desired drug. The use of substituents to ensure that the drug remains in a soluble form in varied pH's and to aid permeation in the digestive track, results in the molecular weight of the modified drug to be enhanced from 1.05 fold to typically 10 fold. However notwithstanding it's increased molecular weight and complexity there is an increase in its rate of absorption and its retention in the blood stream defying the principles of natural chemistry and pharmacology and beautifully reveals the inventive concept and novelty of my invention as normally drugs with such high molecular weights and complexity would normally never traverse the digestive track. As is seen in my invention a drug modified with molecular weight of above 5000 daltons gets into the blood stream much more faster and effectively (in larger quantities) and is retained in the blood stream for more time for performing its role more effectively than it's unmodified parent which has a molecular weight of only around 500 daltons but with a poor solubility.

To understand the present invention certain drugs and pharmaceuticals are taken as examples for the purpose of reference only. Therefore it should not be treated that the reference examples limit the scope of the invention.

In the present invention it should be understood that the unmodified pharmaceutical or drug includes any essential or non-essential amino acid albeit the amino acid is used as further substituent of any other pharmaceutical or drug as defined herein. It is further to be understood that the amino acid as defined as a pharmaceutical or drug includes a polymeric form that is a peptide of smaller length preferably comprising 2 to 10 amino acids bonded through peptide bonds. Therefore an unmodified pharmaceutical or drug is any essential or non-essential amino acid or a precursor of such amino acid or a derivative of such amino acid or a pre-form of such amino acid which is capable of releasing such amino acid as a metabolite either in the digestive tract or at a desired site.

Examples of drugs or pharmaceuticals can be selected from any solid organic molecules with less solubility in aqueous/non aqueous solutions. The preferred drugs and their US patent numbers mentioned in brackets are selected from Atorvastatin (U.S. Pat. No. 5,273,995), Amlodipine (U.S. Pat. No. 4,572,909), Cetirizine (U.S. Pat. No. 4,525,358), Cetraxate (U.S. Pat. No. 3,699,149), Fluticasone, Salmeterol, Omeprazole (U.S. Pat. No. 4,255,431) and derivatives thereof.

### Application of My Invention on Statins:

Hypercholesterolaemia is a risk factor for the development of atherosclerotic disease. Statins like atorvastatin lowers plasma low-density lipoprotein (LDL) cholesterol levels by inhibition of HMG-CoA reductase All statins are not the same—either in terms of efficacy or in their propensity for drug-drug interactions. All statins have the capacity for severe toxicity, including something called rhabdomyolysis (basically, breakdown of muscle fibres resulting in the release of muscle fibre contents into the circulation. This can put undue stress on the kidneys and results in kidney damage) and hepatic dysfunction, which is dose-dependent. Understanding the metabolism of the statin drugs and modifying statins to a safer form will enable synergy between safety and bioavailability.

Certain statins possess properties that limit their hepatic bioavailability, thus decreasing their therapeutic effect and potentially increasing their systemic exposure. The inability to cross biological membranes by diffusion, for example, is one such property. Following ingestion, statins are absorbed through the intestine into the hepatic portal vein and distributed into the liver, which are the primary site of action and the primary site of cholesterol synthesis. Statin compounds that are lipophobic, and/or have high molecular weights often show poor diffusive permeability across biological membranes in vivo. Accordingly, transport across biological membranes is only possible via a carrier mediated transport mechanism that typically requires energy, often supplied by the hydrolysis of ATP.

Improved solubility of statins can achieve a similar efficacy at lower dosage thereby obviating potential adverse side effects (e.g. muscle wasting) associated with these drugs and/or cause the statins to be significantly more anti-inflammatory at any given dose.

### Application of My Invention on Atorvastatin:

To understand the present invention certain drugs and pharmaceuticals are taken as examples for the purpose of reference only. Therefore it should not be treated that the reference examples limit the scope of the invention. In U.S. patent [R—(R*,R*)]-2-(4-fluorophenyl)-.beta.,.delta.-dihydroxy-5-((1-methylethyl)-3-phenyl-4-[(phenyl amino)-carbonyl]-1H-pyrrole-1-heptanoic acid or (2R-trans)-5-(4-fluorophenyl)-2-(1-methylethyl-N,4-diphenyl-1-[2-(tetrahydro-4-hydroxy-6-oxo-2H-1-pyran-2-yl)ethyl]-1H-pyrrole-3-carboxamide and pharmaceutically acceptable salts thereof have been disclosed. This patent is silent about the preparation of liquid formulations. This drug is also known as Atorvastatin that is a solid and has less solubility in aqueous solutions. This drug is found to provide inhibition of the biosynthesis of cholesterol. A need was felt to formulate this drug in liquid form. This need is achieved through the present invention as described hereafter in the description.

Atorvastatin is hereby taken as one of the examples to explain the invention in a vivid manner, however this example cannot be construed as a limiting example to restrict the scope of the invention.

Atorvastatin is poorly water-soluble. For example, as defined in the U.S. Pharmacopia (2002), atorvastatin is considered “very slightly soluble.”

As like all carrier-mediated transport statins, once atorvastatin passes out of the stomach, it is absorbed in the intestine and then in the liver via carrier-mediated transport mechanisms. Only about 30% of orally administered atorvastatin is absorbed from the intestine. Similar to most other statins, atorvastatin undergoes extensive first-pass metabolism (in the liver). About a portion of the atorvastatin absorbed from the intestine is taken up by the liver, resulting in a systemic bioavailability of the parent drug of approximately 14%.

The clinical dosage range for atorvastatin is 10-80 mg/day. However, atorvastatin is subject to extensive first-pass metabolism in the gut wall as well as in the liver, as oral bioavailability is 14%. In vivo, cytochrome P450 (CYP) 3A4 is responsible for the formation of two active metabolites from the acid and the lactone forms of atorvastatin. Metabolism of atorvastatin acid and lactone by human liver microsomes results in para-hydroxy and ortho-hydroxy metabolites. Both substrates were metabolized mainly by CYP3A4 and CYP3A5. Atorvastatin lactone has a significantly higher affinity to CYP3A4 than the acid. A lactone is a cyclic ester in chemistry. It is the condensation product of an alcohol group and a carboxylic acid group in the same molecule Compared with atorvastatin acid, CYP-dependent metabolism of atorvastatin lactone to its para-hydroxy metabolite was 83-fold higher and to its ortho-hydroxy metabolite was 20-fold higher. Since Atorvastatin is a synthetic inhibitor of 3-hydroxy-3-methylglutaryl-coenzyme A (HMG-CoA) reductase, which catalyzes the conversion of HMG-CoA to mevalonate, the rate-limiting step in de novo cholesterol synthesis, we need to ensure that atorvastatin is safely available without its undergoing a change to its lactone form. Linking an aminoacid or a hydroxy polyacid to the carboxylic acid or hydroxyl group(s) of the atorvastatin ensures that there is no scope for formation of its lactone. Since only the lactone form has a higher affinity to react with CYP3A4, elimination of the formation of lactone will result in a very low amount of atorvastatin being metabolized by CYP3A4. As discussed, enzyme CYP3A4 is responsible for metabolising atorvastatin acid to its para and ortho hydroxy forms. This reaction can be curtained or culled if an alkyl, preferably lower alkyl, most preferably a methyl donor is made available at the appropriate location in the molecule. Alkyl groups being electron donors affect the ortho and para portions of the benzene ring to be substituted by —OH group. To prevent the lactonisation of atorvastatin either the free carboxylic group or the beta and delta hydroxy groups, severely or together are modified by reacting with substituents or protected by known groups which during metabolic reactions at liver able to convert back to free hydroxyl groups.

In the present invention as an example the drug or pharmaceutical molecule is treated with acid followed by reacting further with a relevant anion radical to form a complex compounds, which have a high solubility in aqueous/nonaqueous phase. In another embodiment the drug or pharmaceutical molecule containing at least one free acid group (—COOH) and/or hydroxyl/amine group is reacted with alcohol groups like 2-Aminoethanol and further with any known relevant anion radical. Oil soluble drugs are obtained by linking a long chain fatty acid to the drug amide complex or the drug ester complex with the proviso that the amide/ester has additional reactive sites for reacting with the fatty acid.

Atorvastatin has one free carboxylic group at one end, two hydroxy groups and an amine group. These reactive groups can be reacted with either one of the amino groups of amino acids, or organic acids like beta hydroxytricarballyllic acid and in turn with Hydroxy alkyl amines like 2-Aminoethanol to increase the number of reactive sites and finally link with solubilizing enhancers like (Z)-9-Octadecenoic acid (for oil phase) or Betaine (for water phase) to make the final product oil or water soluble.

The inventor of the present invention has surprisingly found that the solubility and in turn bioavailability of Atorvastatin has increased many folds despite the increase in complexity of the molecule.

These observations lead into the present invention, later extended to various less soluble drugs and found most suitable method for achieving improvement in solubility in the varied pHs of the digestive track resulting in improved bioavailability and better retentivity in the blood stream to perform its function better.

In further experiments the same Atorvastatin is reacted with a hydroxyl polyacid by known methods in the art. Further treatment with relevant anion radical(s) as explained above, is responsible for the formation of final complex which was also found to be completely soluble.

### Application of My Invention on Phytosterols:

Since the 1950's numerous studies in animals and humans have been reported, in which plant sterols (phytosterols) have caused significant reductions in serum cholesterol levels. Phytosterols are plant sterols structurally similar to cholesterol that have been known for many years to reduce cholesterol absorption and serum cholesterol levels while not being absorbed themselves. Chemically, natural sterols are C.sub.26-C.sub.30 steroid alcohols, which have an aliphatic side chain at the C.sub.17 position. The differences between a cholesterol molecule and a phytosterol molecule are primarily found in the structure of the side chain of the basic frame. Plant sterols may also be hydrogenated to produce plant stanols, i.e., phytostanols. Plant sterols reduce serum cholesterol levels by reducing the absorption of cholesterol from the digestive tract. Phytosterols are a group of compounds structurally very similar to cholesterol. However unlike cholesterol, they are virtually nonmetabolisable. They compete with cholesterol at the point of absorption and thus reduce its entry into the blood stream. The phytosterols occurring most frequently in the nature are sitosterol, campesterol and stigmasterol. In recent years plant sterol treatment of hypercholesterolemia has been refined by the use of the fully saturated form of sitosterol and sitostanol. Saturated phytosterols such as sitostanol and campestanol are present in our diet in small amounts. Sitostanol is virtually unabsorbed and lowers the cholesterol content of mixed micelles more efficiently than sitosterol, thus showing an enhanced serum cholesterol lowering effect.

During fat digestion dietary fat, sterol and/or stanol together with dietary cholesterol reach the intestinal oil phase (in intestinal emulsion), The stanols and/or sterols compete with both dietary and bileary cholesterol for micellar solubility and lower the micellar phase concentration of cholesterol when present in lipid core fat material of the mixed micelles in high enough concentrations. Plant stanols like sitostanol is more effective in lowering micellar phase cholesterol than the corresponding sitosterol. The solubility of free sterol and especially of free stanol in edible oils and fats is very low. This problem was overcome to a limited extent by esterifying the free sterols with fatty acid esters. However this helped in improving the solubility only to a limited extent For example it was identified that only 0.1%, 0.6% and 1.1% of beta sitosterol was soluble in triolein oil when linked as an ester to palmitic, lauric and decanoic acids. This problem can be overcome in total by the application of the present invention. A number of long chain fatty acid which aid in increasing the solubility of beta sitosterol in oil phase can be linked to beta sitosterol with the aid of a multi carboxylic secondary alcohol like beta hydroxytricarballyllic acid, and hydroxy alkyl amine like 2-Aminoethanol. Betaine, poly ethylene glycol etc can be used instead of the long chain fatty acid mentioned above to get a sterol/stanol which is soluble in the water phase.

This invention also includes improving the solubility of stanols in an oil phase and can be carried out in the same manner as explained above for sterols and by simple replacement of stanol instead of sterol.

By the term phytosterol is in this specification meant 4-desmethyl sterols, 4-monomethyl sterols, and 4,4-dimethyl sterols (triterpene alcohols) or their blends. By the term phytostanol is in this specification meant 4-desmethyl stanols, 4-monomethyl stanols and 4,4-dimethyl stanols preferably obtained by hydrogenation of the corresponding phytosterol. Typical 4-desmethyl sterols are sitosterol, campesterol, stigmasterol, brassicasterol, 22-dehydrobrassicasterol, .DELTA.5-avenasterol. Typical 4,4-dimethyl sterols are cycloartenol, 24-methylenecycloartanol and cyclobranol. Typical phytostanols are sitostanol, campestanol and their 24-epimers, cycloartanol and saturated forms obtained by saturation of triterpene alcohols (cycloartenol, 24-methylenecycloartanol and cyclobranol). By the terms phytosterols and phytostanol in this specification is further meant all possible natural blends of 4-desmethyl sterol and stanols, 4-monomethyl sterols and stanols, 4,4-dimethyl sterols and stanols and mixtures of natural blends. By the terms phytosterols and phytostanols in this specification is further meant any individual 4-desmethyl sterol, 4-monomethyl sterol or 4,4-dimethyl sterol or their corresponding saturated forms. The terms plant sterol and plant stanol are used in this specification as synonyms to phytosterol respectively phytostanol. Sterol and stanol shall also mean phytosterol and phytostanol respectively.

### Application of My Invention on CETP Inhibitors:

CETP inhibitors are another class of compounds that are capable of modulating levels of blood cholesterol, such as by raising high-density lipoprotein (HDL) cholesterol and lowering low-density lipoprotein (LDL) cholesterol. It is desired to use CETP inhibitors to lower certain plasma lipid levels, such as LDL-cholesterol and triglycerides and to elevate certain other plasma lipid levels, including HDL-cholesterol and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

The present invention relates to a CETP inhibitor whose solubility has been enhanced multifold better. CETP inhibitors, particularly those that have high binding activity, are generally hydrophobic, have extremely low aqueous solubility and have low oral bioavailability when dosed conventionally. Such compounds have generally proven to be difficult to formulate for oral administration such that high bioavailabilities are achieved. Accordingly, CETP inhibitors must be formulated so as to be capable of providing good bioavailability. Such formulations are generally termed “solubility-improved” forms. One method for increasing the bioavailability of a CETP inhibitor has been forming a solid amorphous dispersion of the drug and a concentration-enhancing polymer. Another method for increasing the bioavailability of a CETP inhibitor is to formulate the compound in a lipid vehicle. Additional methods for increasing the bioavailability of a CETP inhibitor include adsorbing the CETP inhibitor onto a porous substrate and providing a stabilized amorphous form of a CETP inhibitor with a concentration-enhancing polymer. However all these methods involve modifying only the physical characteristics and not the chemical characteristics.

According to my invention Torcetrapib, an CETP inhibitor and oil insoluble drug can be hydrolysed to obtain Des N Propyl torcetrapib and this can be reacted with substituents as explained in the present invention to obtain a modified form of torcetrapib which has higher oil solubility and in turn higher efficacy.

Substituents and their Applicability in My Invention:

Process of drug uptake generally involves absorption through the small intestine by a carrier-mediated transport mechanism, followed by absorption into hepatocytes, also via a carrier-mediated transport mechanism. Access to the site of action of drugs that are dependent on such carrier-mediated mechanisms depends to a large extent on the capacity of the transport mechanism across the membrane. Transport moieties can comprise of fatty acids like Butanoic Acid, Decanoic Acid, Dodecanoic Acid, Heptadecanoic Acid, Hexanoic Acid, Hexadecanoic Acid, Linoleic Acid. Linolenic Acid, Nonanoic Acid, Octanoic Acid, Oleic Acid, Pentanoic Acid, Tetradecanoic Acid, and Undecylenic Acid. Enhancing agents that can be used to increase intestinal uptake of statins include, but are not limited to fatty acids, fatty acid esters, fatty alcohols and amino acids. Fatty alcohols include, but are not limited to, stearyl alcohol, and oleyl alcohol. Fatty acids include, but are not limited to, oleic acid, lauric acid, myristic acid, palmitic acid, stearic acid, linoleic acid, capric acid, monoglycerides, diglycerides, acylcholines, caprylic acids, acylcarnitines, sodium caprate, and palmitoleic acid. Fatty acid esters include those containing more than 10 to 12 carbons. Examples of fatty acids esters include, but are not limited to, isopropyl myristate and methyl and ethyl esters of oleic and lauric acid. Another group of enhancing agents includes low molecular weight alcohols. Examples of such alcohols include, but are not limited to ethanol, propanol, isopropanol, butanol, benzyl alcohol, glycerin, polyethylene glycol, propanediol and propylene glycol. Amino acids include, but are not limited to Alanine, Arginine, Asparagines, Aspartic Acid, Cysteine, Cystine, Glutamine, Glutamic Acid, Gbetaine, Histidine, Hydroxyproline, Isoleucine, Leucine, Lysine, Methionine, Ornithine, Phenylalanine, Praline, Serine, Threonine, Tryptophan, Tyrosine, Valine and combinations thereof. The list also includes Peptides and Polypeptides formed by any combination of the naturally above occurring amino acids.

In certain embodiments, any of the peptides described herein can be attached to one or more biotin. The biotin interacts with the intestinal sodium-dependent multivitamin transporter and thereby facilitates uptake and bioavailability of orally administered peptides.

The alcohol groups used in the present invention is selected from the alcohols preferably having carbon atoms from 1 to 30 and may define monohydroxy, dihydroxy, trihydroxy or polyhydroxy but preferably either mono di or tri-hydroxyl alcohols and selected from Ethanol, Sorbitol, Manitol, Glucose, Propanol, Butanol, Pentanol, Hexanol, Heptanol, Octanol, Propylene Glycol, Glycerine, Poly Ethylene Glycol, ethanolamine etc and a combination thereof.

The Relevant Anion Radical according to the present invention should be understood as essentially a molecule capable of releasing a positive ion such as hydrogen ion, capable of accepting the lone pair of electrons and capable of forming dative bond or bonds. The molar ratio of the drug molecule to amino acids and to Relevant Anion Radical is not critical for the manufacture of improved organic compounds of the present invention. According to the present invention it is possible to manufacture a composition comprising a combination of drug molecules bonded with different organic bases and/or with different relevant anion radicals. Linking a transport moiety/enhancing agent/buffer to a drug or pharmaceutical would aid in the modified drug or pharmaceutical to reach the bloodstream faster, safer and more effectively. Efforts have been made by others earlier to link one or two substituents to the drug or pharmaceutical thereby improving the desired characteristics very mildly. My invention ensures that the characteristics of the drug or pharmaceutical are completely modified to the desired level by using the desired number of substituents which act as transport moieties, enhancers and/or buffers. Presence of buffers linked to the drug or pharmaceutical ensures the drug or pharmaceutical to remain in a soluble form and in turn in a bioabsorbable form without precipitating.

As observed, the earlier discoveries have been made to improve the solubility of a drug by changing the physical characteristics. Changing the physical characteristics include identifying crystalline/amorphous forms of an established drug with better solubility, using surfactants to reduce surface tension and increase solubility, changing the pore size/particle size to help form better dispersions, and using various mixtures of solvents, colloidal agents, wetting agents etc. to improve drugs solubility, also using the aid of modern equipments like homogenizers to disperse the active drug in a formulation better.

Nowhere has the chemical structure of the active drug been modified to vastly improve its solubility in aqueous/non aqueous media. The present invention is thus unique, different from other discoveries made till date. By linking an active ingredient with permeation enhancers, which also aid in improving solubility of a drug in the aqueous/non aqueous media, the bio absorption of a drug is vastly improved. The advantage of chemical linking the permeation enhancers with the active drug components is that the permeation enhancers are positioned at a molecular level nearest to that of the drug and ensure the better bio absorption of the drug. The drug chemical bonded with permeation enhancers is obviously more stable in digestive tract than the drug whose physical characteristics have been modified merely through physical means as logically a permeation enhancer when physically mixed would be absorbed quicker leaving the drug or pharmaceutical behind. Contrarily a chemical bond between the drug or pharmaceutical and enhancer cum buffer would ensure that the drug or pharmaceutical is in a soluble form and traverses the digestive track at the same speed as that of the enhancer. More the number of substituents attached to the drug or pharmaceutical, better would be the performance.

### OBJECTS

The foremost object of the invention is to modify the least soluble drug compounds into soluble forms both in non aqueous solutions like fats, lipids etc., and/or aqueous solutions and enhancing the bioavailability of drugs including its compositions, salts, solvates and a combination thereof. Therefore the first object of the invention is to modify the less soluble drug or pharmaceutical molecules into highly and completely soluble forms so as to provide high bioavailability and bioabsorbability. The second object of the present invention is to provide less soluble drugs or pharmaceuticals in liquid forms for oral administration.

The third object of the invention is to provide the less soluble drug or pharmaceuticals in liquid/gel form for topical administration.

The fourth object of the invention is to provide the less soluble drug or pharmaceuticals in suitable liquid forms for intra veinous or sub cutaneous administrations.

The fifth object of the invention is to provide less soluble drugs or pharmaceuticals in a form such that it has higher retentivity in the blood stream.

The sixth object of the invention is to provide the drug or pharmaceutical in a more effective form such that the same amount of active drug or pharmaceutical performs better ensuring use of same dosage equivalent for getting better performance or use of lesser dosage for getting same performance.

The seventh object of the invention is to further enhance the solubility of an improved drug or pharmaceutical compound in the presence of cosolubilisers.

Accordingly the present invention relates to improved pharmaceutical compounds wherein the less soluble drugs or pharmaceuticals is modified into highly or completely soluble either in aqueous or non-aqueous solvents or solutions, formulating the said solutions in a known manner for oral administration or for intra veinous or sub cutaneous administrations.

The present invention also relates to a process of preparing the improved pharmaceutical compounds having high solubility or complete solubility wherein the less soluble drugs or pharmaceuticals are first treated with acids and optionally treated with relevant anion radical(s) under suitable conditions.

The present invention also relates to a process of preparing the improved pharmaceutical compounds having high solubility or complete solubility wherein the less soluble drugs or pharmaceuticals having at least one carboxylic acid/hydroxyl/amine group wherein at least one group is reacted with a bonded combination of permeation enhancers and buffers to ensure that the modified forms traverse the digestive track and reaches the bloodstream the fastest, safest and most effective manner.

In further experiments it was surprisingly found by the inventor of the invention that in the presence of cosolubiliser the solubility of improved drug or pharmaceutical compounds of the present invention is still enhanced.

These observations lead into the present invention, later extended to various less soluble drugs and found most suitable method for achieving improvement in solubility in the varied pHs of the digestive track resulting in improved bioavailability and better retentivity in the blood stream to perform its function better.

The invention also includes amorphous & polymorph modifications of drug or pharmaceutical and formulations containing them.

It is to be construed that the permutations and combinations of the present invention, which might not have been mentioned in this description, is also a part of the invention.

The following examples further illustrate the invention described herein and are in no way intended to limit the scope of the invention. The reactions explained below are preferably carried out at ambient temperatures and pressures.

### EXAMPLES

### Example 1

### Preparation of an Oil Soluble Form of Atorvastatin

Atorvastatin calcium (the form commercially available) is added to a 5% solution of Sodium EDTA in water, in a round bottom flask and stirred briskly. The Sodium EDTA chelates the calcium and renders atorvastatin in its open acid form. Atorvastatin acid being water insoluble is extracted and dried.

1 gram mole equivalent of the atorvastatin extracted as above is digested in 100 ml of ethyl acetate in a round bottom flask fitted with a magnetic stirrer. 1 gram mole equivalent of (S)-2,6-diaminohexanoic acid is added to the above ethyl acetate solution and stirred at ambient room temperature. The reactants, which were initially insoluble in ethyl acetate slowly, react and form atorvastatin (S)-2,6-diaminohexanoic acid complex, which is completely soluble in ethyl acetate solution. The solution of clear ethyl acetate containing atorvastatin (S)-2,6-diaminohexanoic acid complex is then poured into a glass tray and the ethyl acetate is allowed to evaporate till the atorvastatin (S)-2,6-diaminohexanoic acid complex is obtained as a dry product.

1 gram mole equivalent of the dry atorvastatin (S)-2,6-diaminohexanoic acid complex obtained as above is dissolved in 200 ml of carbon tetra chloride in a round bottom flask fitted with a magnetic stirrer. To this is added 1 gram mole equivalent of acyl chloride of (Z)-9-Octadecenoic acid and the solution is stirred for 4 hrs. After completion of the reaction the above solution is poured into the glass tray to allow the carbon tetrachloride to evaporate. The atorvastatin (S)-2,6-diaminohexanoic acid (Z)-9-Octadecenoic complex as obtained above is washed with a 5% solution of sodium carbonate in a separating funnel to neutralize the hydrochloride present in the product. After washing the atorvastatin (S)-2,6-diaminohexanoic acid (Z)-9-Octadecenoic complex is obtained as a separate oily layer and is separated from the saline sodium carbonate water solution. The final atorvastatin (S)-2,6-diaminohexanoic acid (Z)-9-Octadecenoic complex obtained is an oily liquid freely soluble in oils including arachis oil, soya bean oil etc.,

### Example 2

### Preparation of Water-Soluble Form of Atorvastatin Namely Atorvastatin (S)-2,6-diaminohexanoic Acid Betaine

Atorvastatin calcium (the form commercially available) is added to a 5% solution of Sodium EDTA in water, in a round bottom flask and stirred briskly. The Sodium EDTA chelates the calcium and renders atorvastatin in its open acid form. Atorvastatin acid being water insoluble is extracted and dried.

1 gram mole equivalent of the atorvastatin extracted as above is digested in 100 ml of ethyl acetate in a round bottom flask fitted with a magnetic stirrer. 1 gram mole equivalent of (S)-2,6-diaminohexanoic acid is added to the above ethyl acetate solution and stirred at ambient room temperature. The reactants, which were initially insoluble in ethyl acetate slowly, reacts with the atorvastatin acid and forms atorvastatin (S)-2,6-diaminohexanoic acid complex which is completely soluble in ethyl acetate solution. To this solution is added 1 gram mole equivalent of betaine and stirring continued till a precipitate of atorvastatin (S)-2,6-diaminohexanoic acid betaine, which is insoluble in ethyl acetate, is obtained. This precipitate Atorvastatin (S)-2,6-diaminohexanoic acid betaine is spread over in a glass tray to allow the ethyl acetate to evaporate. This atorvastatin (S)-2,6-diaminohexanoic acid betaine has a solubility of 100 mg in 100 ml of water and 1 gm in 5 ml of lactic acid. As is observed the solubility of atorvastatin (S)-2,6-diaminohexanoic acid betaine in water is far superior to that of atorvastatin calcium whose solubility is documented as being “very slightly soluble” as per (US pharmacopia 2002). As per the US Pharmacopia norms “very slightly soluble” refers to aqueous solubility that ranges from 1/1000 to 1/10,000 mg per ml. Thus it is observed that solubility of atorvastatin (S)-2,6-diaminohexanoic acid betaine has been increased a 1000-10000 folds higher over that of atorvastatin calcium.

### Example 3

### Preparation of an Oil Soluble Form of Atorvastatin (Atorvastatin Oil), which Also Remains as a Solution without Precipitating in the Whole Range of pH's as Found in the Digestive Tract of an Organism

Atorvastatin calcium (the form commercially available) is added to a 5% solution of Sodium EDTA in water, in a round bottom flask and stirred briskly. The Sodium EDTA chelates the calcium and renders atorvastatin in its open acid form. Atorvastatin acid being water insoluble is extracted and dried.

1 gram mole equivalent of beta hydroxytricarballylic acid is reacted with 3 gram moles equivalent of sulfurous oxychloride dissolved in 100 ml of tetrahydrofuran in a round bottom flask at ambient room temperature.

1 gram mole equivalent each of 2-aminoethanol, 2-aminoethanol (z)-9-octadecenoiate and 2-aminoethanol bis(z)-9-octadecenoiate are added to the above solution in the round bottom flask. Evolution of hydrochloric acid takes place and all the forms of aminoethanol mentioned above completely reacts with the beta hydroxytricarballylic acid. The product obtained above is labeled as reactant-1 of this example.

Next 1 gram mole equivalent of beta hydroxytricarballylic acid is dissolved in tetrahydrofuran and reacted with 3 gram mole equivalents of sulfurous oxychloride. Stirring is continued till the reaction is completed. Next added simultaneously the contents of reactant-1 of this example, 1 gram mole equivalents of 2-aminoethanol (z)-9-octadecenoiate and 0.33 gram mole equivalents of atorvastatin acid. The solution is stirred for 3 hours for completion of reaction.

The reactant obtained above is poured on a glass tray and left open at room temperature till all the solvent evaporates. An oily product of atorvastatin is obtained and will be labeled atorvastatin oil in future for simplicity. This oily product is highly soluble in arachis oil, sunflower oil, soybean oil etc.

### Example 4

### Preparation of an Oil Soluble Form of Rosuvastatin, which Also Remains as a Solution without Precipitating in the Whole Range of pH's as Found in the Digestive Tract of an Organism

Rosuvastatin calcium (the form commercially available) is added to a 5% solution of Sodium EDTA in water, in a round bottom flask and stirred briskly. The Sodium EDTA chelates the calcium and renders rosuvastatin in its open acid form. Rosuvastatin acid being water insoluble is extracted and dried.

1 gram mole equivalent of beta hydroxytricarballylic acid is reacted with 3 gram mole equivalents of sulfurous oxychloride dissolved in 100 ml of tetrahydrofuran in a round bottom flask at ambient room temperature.

1 gram mole equivalent each of 2-aminoethanol, 2-aminoethanol (z)-9-octadecenoiate and 2-aminoethanol bis(z)-9-octadecenoiate are added to the above solution in the round bottom flask. Evolution of hydrochloric acid takes place and all the forms of aminoethanol mentioned above completely reacts with the beta hydroxytricarballylic acid. The product obtained above is labeled as reactant-1 of this example.

Next 1 gram mole equivalent of beta hydroxytricarballylic acid is dissolved in tetrahydrofuran and reacted with 3 gram mole equivalent of sulfurous oxychloride. Stirring is continued till the reaction is completed. Next simultaneously added the contents of reactant-1 of this example, 1 gram mole equivalent of 2-aminoethanol (Z)-9-octadecenoiate and 0.5 gram mole equivalent of rosuvastatin acid. The solution is stirred for 3 hours for completion of reaction.

The reactant is poured on a glass tray and left open at room temperature till all the solvent evaporates. An oily product of rosuvastatin is obtained and will be labeled rosuvastatin oil in future for simplicity This oily product is highly soluble in arachis oil, sunflower oil, soybean oil etc.

### Example 5

### In Vitro Studies to Establish the Solubility of Atorvastatin Oil and Rosuvastatin Oil in pH Conditions Ranging from 1 to 8, Simulating the pH Conditions of the Digestive Tract of an Organism

0.5 gram of atorvastatin oil and rosuvastatin oil as synthesized in examples 3 and 4 above were independently mixed with 3 gms of polyoxyl 40 hydrogenated castor oil at 60° C. and diluted in a solution containing 16.5 ml water kept at a temperature of 60° C. A clear homogenous solution of atorvastatin and rosuvastatin in water was thus obtained. The pH of these individual solutions were reduced to 1 by addition of hydrochloric acid and the solutions were observed to be clear without any precipitation to indicate that the atorvastatin oil and rosuvastatin oil are very stable in the acidic conditions. Next the pH of the solutions were increased up to 8 by addition of Sodium bicarbonate. It was observed that the solutions remained completely clear indicating that atorvastatin oil and rosuvastatin oil were also stable in the alkaline conditions of the digestive tract. Enhanced stability as above in the whole range of pH's of the digestive track ensures complete assimilation without the drug being precipitated in the digestive tract, an important criteria to ensure enhanced bio absorbability.

### Example 6

### Pharmacokinetic Profile of Test Substance Namely Atorvastatin Oil (Synthesized as Per Example 3 Stated Above), in Animal Model Namely Rabbit

### OBJECTIVE

To characterize the atorvastatin oil (synthesized as per example 3 stated above, herein referred to as the Test substance) for pharmacokinetic parameters 1) Cₘₐₓ ii) Tₘₐₓ iii) AUC 0-α iv) AUMC 0-α v) Mean Residence Time (MRT), when given by oral route of administration in rabbits.

### Materials:

Atorvastatin calcium—reference standard. Test substance—atorvastatin oil, Tween 80, heparin. HPLC grade solvents—acetonitrile, water.

### Animals:

Healthy male/female rabbits of weight 1-1.5 kgs, age 3 months.

### Procedure:

12 animals were divided into groups of six animals each. Group I received atorvastatin calcium as reference drug and Group II received atorvastatin oil as the test substance.

DOSE: 2.4 mg equivalent of atorvastatin/kg body weight to both control and test group of rabbits.

ROUTE: Oral route of administration

Procedure details: To Group I (rabbits 1 to 6), a suspension of the standard i.e. atorvastatin calcium in Tween 80 was administered. To Group II (rabbits 7 to 12), test substance i.e. atorvastatin oil synthesized as per example 3 stated above was provided as a water soluble liquid, by solubilizing the oil soluble form in Tween 80 and administered as such. 0.5-1 ml of blood was with drawn from the marginal ear vein at the following time intervals after drug administration.

0, 15, 30, 45, 60, 90, 120, 180, 240, 360, 480 mts, 24 hrs i.e. 12 samples/rabbit.

Estimation of Atorvastatin by HPLC method:

Conditions:

| | |
|---|---|
| Column | ODS, C 18 (250 mm × 4.5 mm, 5 μ) |
| Flow rate | One ml/min |
| Mobile phase | Acetonitrile:water (70:30) |
| Retention time | 2.4 mts (approx) |
| Volume load | 20 μl |
| Detection - uv | 250 nm |
| Diluent | same as mobile phase |

Whole blood withdrawn was centrifuged to separate serum. From the clear serum, 0.1 ml was pipetted and deproteinated and diluted with 0.4 ml of acetonitrile. The clear supernatant was used for estimation.

CONCENTRATION OF ATORVASTATIN (WHEN DOSED AS
ATORVASTATIN CALCIUM) IN BLOOD PLASMA AS DETERMINED IN
STANDARD - GROUP 1
Concentration of atorvastatin
(when dosed as atorvastatin calcium)
in blood plasma Units: mcg/ml

| Sampling | | | | | | | |
|---|---|---|---|---|---|---|---|
| time | Rabbit 1 | Rabbit 2 | Rabbit 3 | Rabbit 4 | Rabbit 5 | Rabbit 6 | Average |
| 15 min | 15.73 | 12.80 | 14.50 | 13.85 | 12.50 | 16.10 | 14.25 |
| 30 min | 40.90 | 37.65 | 39.52 | 38.42 | 37.50 | 41.25 | 39.21 |
| 45 min | 51.05 | 46.40 | 50.45 | 48.75 | 47.20 | 52.25 | 49.35 |
| 60 min | 71.28 | 67.90 | 70.35 | 69.23 | 68.53 | 72.54 | 69.97 |
| 1½ hr | 103.05 | 95.05 | 100.80 | 98.70 | 96.14 | 105.40 | 99.86 |
| 2 hr | 122.60 | 116.00 | 120.75 | 119.62 | 117.50 | 124.55 | 120.17 |
| 3 hr | 130.61 | 125.10 | 133.02 | 128.90 | 127.10 | 131.65 | 129.40 |
| 4 hr | 118.27 | 122.60 | 120.40 | 124.50 | 123.55 | 120.51 | 121.64 |
| 6 hr | 101.05 | 106.05 | 105.52 | 104.30 | 103.75 | 106.25 | 104.49 |
| 8 hr | 71.63 | 78.10 | 75.40 | 77.50 | 76.54 | 73.50 | 75.45 |
| 24 hr | 40.40 | 45.50 | 42.70 | 41.20 | 44.30 | 43.50 | 42.93 |

Cₘₐₓ=129.4 mcg/ml Tₘₐₓ=180 mts (3 hrs)
AUC 0-α=177192 mcg-min/ml
AUMC 0-α=288437978 mcg-min*min/ml

$$Mean Residence Time ( M R T ) = \frac{AUMC 0 - α}{AUC 0 - α} = 1627.8 MINUTES$$

CONCENTRATION OF ATORVASTATIN (WHEN DOSED AS
ATORVASTATIN OIL SYNTHESIZED AS PER EXAMPLE 3) AS
DETERMINED IN TEST - GROUP II
Concentration of atorvastatin
(when dosed as atorvastatin oil)
in blood plasma Units: mcg/ml

| Sampling | | | | | | | |
|---|---|---|---|---|---|---|---|
| time | Rabbit 7 | Rabbit 8 | Rabbit 9 | Rabbit 10 | Rabbit 11 | Rabbit 12 | Average |
| 15 min | 92.66 | 94.50 | 98.50 | 47.57 | 93.75 | 94.40 | 86.90 |
| 30 min | 132.64 | 135.75 | 140.01 | 61.06 | 130.51 | 132.75 | 122.12 |
| 45 min | 145.07 | 146.80 | 147.50 | 98.81 | 140.61 | 145.75 | 137.42 |
| 60 min | 160.64 | 165.75 | 164.52 | 123.77 | 162.67 | 162.77 | 156.69 |
| 1½ hr | 184.08 | 190.54 | 188.08 | 150.52 | 180.52 | 189.55 | 180.55 |
| 2 hr | 152.61 | 155.65 | 150.71 | 120.51 | 153.63 | 150.45 | 147.26 |
| 3 hr | 131.05 | 135.60 | 130.50 | 115.60 | 131.21 | 130.51 | 129.08 |
| 4 hr | 110.65 | 112.75 | 113.51 | 100.52 | 100.75 | 108.51 | 107.78 |
| 6 hr | 92.15 | 95.25 | 91.75 | 85.54 | 89.90 | 93.40 | 91.33 |
| 8 hr | 59.65 | 63.75 | 60.60 | 60.75 | 58.15 | 59.75 | 60.44 |
| 24 hr | 43.15 | 46.20 | 44.51 | 40.51 | 42.51 | 44.52 | 43.57 |

Cₘₐₓ=180.6 mcg/ml
Tₘₐₓ=90 mts (1½ hrs)
AUC 0-α=230500 mcg-min/ml
AUMC 0-α=613850487 mcg-min*min/ml

$$Mean Residence Time ( M R T ) = \frac{AUMC 0 - α}{AUC 0 - α}$$

MRT=2663 MINUTES

### Comments:

Test substance is absorbed faster when compared to standard, as Tₘₐₓ for test is 1½ hr and for standard is 3 hrs. Cₘₐₓ for test is higher than that for standard (180.6 mcg/ml for test, 129.4 mcg/ml for standard). Test substance absorption is more by 30% and it also stays for longer time, mean residence time—2663 minutes for test against 1627.8 minutes for standard.

1. A modified pharmaceutical or drug exhibiting the synergistic properties of enhanced bioavailability, enhanced bioabsorbability, enhanced solubility at various pH's preferably from high acidic levels to milder basic levels, enhanced absorptivity (increased rate of absorption), decreased toxicity and enhanced retentivity of it's predecessor in the blood, despite it's increased molecular complexity.

2. A modified pharmaceutical or drug as claimed in claim 1 has plurality of additional substituents than the actual active pharmaceutical or drug component, wherein the said substituents are capable of enhancing the solubility in aqueous and/or non-aqueous solvents/medium, capable of enhancing the bioavailability and bioabsorbability, capable of remaining in solution without precipitating at a varied pH levels of the digestive tract, capable of enhancing the rate of absorption and capable of enhancing retentivity in the blood stream.

3. A modified pharmaceutical or drug as claimed in claim 1 has plurality of additional substituents than the actual active pharmaceutical or drug component, wherein the said substituents have optionally additional active sites for further complexing the compound.

4. A modified pharmaceutical or drug as claimed in claim 1 whose solubility in aqueous/non aqueous phase is vastly enhanced by firstly linking the required reactive site(s) of the unmodified drug or pharmaceutical with substituents optionally containing further plurality of reactive sites and secondly linking with further substituents which aid in improving desired solubility, to this reactive sites, the reactive sites containing amongst. Oxygen, Sulphur and/or Nitrogen elements present with/without a lone pair of electron present inside/outside the nucleus.

5. A modified pharmaceutical or drug as claimed in claim 1 prepared from an active pharmaceutical or drug component comprising the active sites, and capable of reacting and forming bond(s) with other chemical entities which when bonded do not alter the mode of action of the said pharmaceutical or drug, wherein the active sites of an unmodified pharmaceutical or drug includes functional groups which comprises atoms like Oxygen, Nitrogen, Sulphur, etc.

6. A modified pharmaceutical or drug as claimed in claim 1 which, notwithstanding it's increased complexity and higher molecular weight than its predecessor, when orally ingested aids in enabling the drug or pharmaceutical to remain in a more soluble form in the varied pH's of the digestive track and to permeate the digestive track faster, safer and more effectively into the blood stream and also retained better ensuring use of it at a lesser dosage for obtaining a desired performance or use of it at the same dosage for obtaining a better performance, the strength of bonding between the pharmaceutical or drug and the substituent being such that after performing the above function the substituent gets detached and metabolized leaving the active drug to perform its role, wherein the reactive sites of the pharmaceutical or drug molecule comprises of Oxygen, Sulphur and/or Nitrogen atoms.

7. A modified pharmaceutical or drug as claimed in claim 1 wherein the said modified pharmaceutical or drug has a plurality of acidic groups when the substituent linked is beta hydroxytricarballylic acid and like, an hydroxyl group when the substituent linked is 2-aminoethanol and like and/or a long chain fatty acid(s) preferably in bonded combination linked to the active sites of unmodified pharmaceutical-compound which exhibits enhanced solubility in the oil phase and helps in permeating through the cell wall at a faster rate.

8. A modified pharmaceutical or drug as claimed in claim 1 includes modified potential inhibitor compounds, such as modified Phytosterol, modified stanols required to minimize the synthesis of undesired excess amount of metabolic substances such as cholesterol.

9. A modified pharmaceutical or drug as claimed in claim 1 wherein the unmodified pharmaceutical or drug is selected from a statin preferably acid form of cerivastatin, hydrolyzed acid form of simvastatin, acid form of pravastatin, acid form of fluvastatin, hydrolyzed acid form of lovastatin, rosuvastatin, pitavastatin, atorvastatin etc., and derivatives thereof, but most preferably atorvastatin, rosuvastatin, pitavastatin, their derivatives and/or a combination thereof.

10. A modified pharmaceutical or drug as claimed in claim 1 wherein the unmodified pharmaceutical or drug is selected from a sterol or stanol including sitosterol, campesterol and stigmasterol, sitostanol and campestanol, their derivatives and/or a combination thereof.

11. A modified pharmaceutical or drug as claimed in claim 1 wherein the unmodified pharmaceutical or drug is selected from a Des N propionyl form of Torcetrapib, Des di N methyl form of Citalopram, Des di N methyl Escitalopram their derivatives and/or a combination thereof.

12. A modified pharmaceutical or drug as claimed in claim 1 wherein the unmodified pharmaceutical or drug is selected from the group of Abacavir, Acetaminophen, Acyclovir, Albuterol, Alendronate, Allopurinol, Alprazolam, Amiodarone, Amitriptyline, Amlodipine, Amoxicillin clavulanate, Anastrozole, Amphetamine, Aripiprazole, Aspirin, Atazanavir, Atropine, Atenolol, Atomoxetine, Azithromycin, Baclofen, Benazepril, Benzonatate, Benztropine, Bicalutamide, Bisoprolol, Budesonide, Butalbital, Bupropion, Captopril, Candesartan, Carbamazepine, Carbidopa, Carisoprodol, Cefdinir, Cefprozil, Celecoxib, Cephalexin, Cetraxate, Cetirizine, Chlorpheniramine, Chlorhexidine, Ciprofloxacin, Citalopram, Carvedilol, Clarithromycin, Clindamycin, Clobetasol, Clonazepam, Clonidine, Clopidogrel, Cyclobenzaprine, Desogestrel, Desloratadine, Diazepam, Diclofenac, Digoxin, Diphenoxylate, Diltiazem, Dolasetron, Donepezil, Doxazosin, Doxepin, Doxycycline, Ebastine, Efavirenz, Enalapril, Erythromycin, Ethinyl Estradiol, Etodolac, Escitalopram, Esomeprazole, Ethinyl Estradiol, Ezetimibe, Famotidine, Felodipine, Fentanyl, Finasteride, Fluoxetine, Fexofenadine, Fluconazole, Fluticasone, Furosemide, Fluvastatin, Folic acid, Fosinopril, Gabapentin, Gatifloxacin, Gemfibrozil, Gemcitabine, Glimepiride, Glipizide, Glyburide, Goserelin, Granisetron, Hydrocodone, Hydroxychloroquine, Hydrochlorothiazide, Hydroxyzine, Hyoscyamine, Ibuprofen, Imatinib, Indapamide, indomethacin, Irbesartan, Ipratropium, Isosorbide, Isotretinoin, Ketoconazole, Labetalol, Lamivudine, Lamotrigine, Lansoprazole, Leuprolide, Levodopa, Levetiracetam, Levofloxacin, Levonorgestrel, Levothyroxine, Linezolid, Lisinopril, Lopinavir, Loratadine, Lorazepam, Lcsartan, Lovastatin, Meclizine, Meloxicam, Mesalamine, Metaxalone, Metformin, Methocarbamol, Methotrexate, Methylphenidate, Methylprednisolone, Metoclopramide, Metoprolol, Mirtazapine, Modafinil, Montelukast, Morphine, Moxifloxacin, Mycophenolate Mofetil, Naproxen, Nelfinavir, Niacin, Nifedipine, Nitrofurantoin, Norethindrone, Norgestimate, Norgestrel, Nortriptyline, Nystatin, Olanzapine, Olmesartan Medoxomil, Olopatadine, Omeprazole, Ondansetron, Oseltamivir, Oxcarbazepine, Oxybutynin, Oxycodone, Oxcarbazepine, Pantoprazole, Paroxetine, Penicillin, Phenazopyridine, Phenobarbital, Phentermine, Phenyloin, Pioglitazone, Prednisone, Promethazine, Propoxyphene, Propranolol, Quinapril, Quinine, Quetiapine, Rabeprazole, Raloxifene, Ramipril, Ranitidine, Risperidone, Risedronate, Ritonavir, Ritonavir, Rofecoxib, Rosiglitazone, Salmeterol, Sertraline, Sevelamer, Sildenafil, Stavudine, Sumatriptan, Sulfamethoxazole, Tadalafil, Tamoxifen, Tamsulosin, Tegaserod, Temazepam, Testosterone, Temozolomide, Teonfovir, Terazosin, Tetracycline, Terbinafine, Thalidomide, Theophylline, Tizanidine, Tobramycin, Tolterodine, Topiramate, Torcetrapeb, Tramadol, Trazodone, Triamterene, Trimethoprim, Valdecoxib, Valacyclovir, Valproic Acid, Valsartan, Venlafaxine, Verapamil, Warfarin, Zidovudine, Ziprasidone, Zolpidem, Zolmitriptan, their derivatives and/or a combination thereof.

13. A modified pharmaceutical or drug as claimed in claim 1 wherein the unmodified pharmaceutical or drug is any essential or non-essential amino acid/peptide or a precursor of such amino acid or a derivative of such amino acid or a pre-form of such amino acid which is capable of releasing such amino acid as a metabolite either in the digestive tract or at a desired site.

14. A modified pharmaceutical or drug as claimed in claim 1 wherein the unmodified pharmaceutical or drug is selected from a group of metal organic chelates preferably aminoacid chelates wherein the metal includes boron, calcium, chromium, copper, cobalt, iron, magnesium, manganese, molybdenum, nickel, potassium, selenium, vanadium, zinc etc.

15. A modified pharmaceutical or drug as claimed in claim 1 is atorvastatin (S)-2,6-diaminohexanoic acid (Z)-9-octadecenoate;
atorvastatin (S)-2,6-diaminohexanoic acid beta hydroxytricarballylate bis 2-aminoethanol di (z)-9-octadecenoiate;
atorvastatin octa (S)-2,6-diaminohexanoic acid hepta betaine;
atorvastatin tetra (S)-2,6-diaminohexanoic acid octa 2-Hydroxypropanoate;
atorvastatin (S)-2,6-diaminohexanoic acid beta hydroxytricarballylate bis 2-aminoethanol tetra (z)-9-octadecenoiate;
atorvastatin (S)-2,6-diaminohexanoic acid bis beta hydroxytricarballylate tetra 2-aminoethanol octa (z)-9-octadecenoiate;
atorvastatin hexa beta hydroxytricarballylate tri 2-aminoethanol hexa 2-aminoethanol (Z)-9-octadecenoiate tri 2-aminoethanoldi (Z)-9-octadecenoiate;
atorvastatin 2-aminoethanol di ethyl beta hydroxytricarballylate;
rosuvastatin tetra beta hydroxytricarballylate di 2-aminoethanol tetra 2-aminoethanol (z)-9-octadecenoiate di 2-aminoethanoldi (z)-9-octadecenoiate;
Ezetimibe tetra beta hydroxytricarballylate di 2-aminoethanol tetra 2-aminoethanol (z)-9-octadecenoiate di 2-aminoethanoldi (z)-9-octadecenoiate;
Des N propyl torcetrapib beta hydroxytricarballylate bis 2-aminoethanol di (z)-9-octadecenoiate;
Betasitosterol beta hydroxytricarballylate bis 2-aminoethanol di (z)-9-octadecenoiate etc.

16. A modified pharmaceutical or drug as claimed in claim 1 is further complexed with Betaine, sarosine or various forms of lysophosphatidyl like Lysophosphatidyl Choline, Lysophosphatidyl Ethanolamine, Lysophosphatidyl Glycerol, Lysophosphatidyl serine, Lysophosphatidic Acid, Lysophosphatides etc. in single or in combination.

17. A modified pharmaceutical or drug as claimed in claim 1 wherein the substituent(s) is/are selected from the group of Acetic Acid, Adipic Acid, Alanine, Arginine, Ascorbic Acid, Arachidonic acid, Asparagines, Aspartic Acid, Betaine, Benzoic acid, Butanol, Butanoic Acid, Carbonic Acid, Capric acid, Citric Acid, Cysetine, Cystine, Decanoic Acid, Dodecanoic Acid, Ethanol, Ethylene glycol, Ethanolamine, Eicosapentanoic Acid (EPA), Folic Acid, Formic Acid, Fumaric Acid, Gluconic Acid, Glucoheptanoic Acid, Glutamine, Glutamic Acid, Glycirine, betaine, Hexanoic Acid, Hexadecanoic acid Heptanoic Acid, Heptadecanoic Acid, Histidine, Hydroxide, Hydrochloric Acid, Hydroxy Proline, Isolencine, Isopropanol, Lactic Acid, Lauryl Sulphonic Acid, Lactobionic Acid, Leucine, Linoneic Acid, Linolenic Acid, Lysine, Malic Acid, Methionine, Mysteric acid, Nicotinic Acid, Nitric Acid, Nonanoic Acid, Octanoic Acid, Octadecanoic Acid, Octanol, Oleic Acid, Oleyl alcohol, Ornithine, Palmitic Acid, palmitoleic acid, Pentanoic Acid, Pentanol, Phenylalanine, Proline, Propanol, Propionic Acid, Propylene glycol, Phosphoric Acid, Retinoic Acid, Sarcosine, Salicylic Acid, Salicylic Acid Acetate, Serline, Selenious Acid, Stearic Acid, Stearyl alcohol, Succinic Acid, Sulphuric Acid, Tartaric Acid, Tetradecanoic Acid, Threonine, Tryptophan, Tyrosine, Undecanoic Acid, Ursodeoxycholic Acid Valine etc., or a combination thereof.

18. A oil soluble modified pharmaceutical or drug as claimed in claim 1 is further converted into an inclusion compound with cyclodextrin.

19. A modified pharmaceutical or drug as claimed in claim 1 is formulated for oral ingestion or for topical use.

20. A process for enhancing the properties like solubility, bioavailability, bioabsorbability of known pharmaceutical or drug having at least one reactable site wherein at least one reactive site is reacted with a pre prepared compound(s) to obtain a modified form of the said known pharmaceutical or drug(s) exhibiting the enhanced properties as defined and claimed above.

21. A process of preparing the pre prepared compound as claimed in claim 20 comprises the steps of:
i) reacting one mole of beta hydroxytricarballyllic acid with three moles of 2-aminoethanol such that two of the 2-aminoethanol links to beta hydroxytricarballyllic acid through an acid amide bond and the remaining one mole of 2-aminoethanol links to beta hydroxytricarballyllic acid through an ester bond leaving amino groups free for further reaction,
ii) treating the product of steps (i) with long chain fatty acid halides for example (z)-9-octadecenoic acid chloride wherein the hydrogens of free amino groups present in the product of step (a) is/are replaced with (z)-9-octadecenoic acid moiety,
iii) treating the product of step (ii) with one mole of beta hydroxytricarballyllic acid to obtain a tertiary amine compound,
iv) further treating the product of step (iii) with long chain fatty acid substitutes at nitrogen of 2-aminoethanol to obtain an acid amide derivative as a pre prepared compound-1.

22. A process for enhancing the properties of solubility, bioavailability, bioabsorbability of atorvastatin wherein one mole of atorvastatin is treated with 3 moles of pre prepared compound-1 as claimed in claim 21.

23. A process for enhancing the properties of solubility, bioavailability, bioabsorbability of rosuvastatin wherein one mole of rosuvastatin is treated with 2 moles of pre prepared compound-1 as claimed in claim 21.

24. A process for enhancing the properties of solubility, bioavailability, bioabsorbability of ezetimibe wherein one mole of ezetimibe is treated with 2 moles of pre prepared compound-1 as claimed in claim 21.

25. A process of preparing the pre prepared compound as claimed in claim 20 comprises the steps of:
i) reacting one mole of (z)-9-octadecenoic acid with one mole of sulfurous oxychloride to produce (z)-9-octadecenonyl chloride; further reacting two moles of (z)-9-octadecenonyl chloride with one mole of 2-aminoethanol to obtain one mole of 2-aminoethanol bis(z)-9-octadecenoiate;
ii) reacting one mole of beta hydroxytricarballyllic acid with three moles of sulfurous oxychloride; further reacting one mole of the product obtained in this step with two moles of the product obtained in step (i) to obtain one mole of acyl chloride of beta hydroxytricarballylate bis 2-aminoethanoldi (z)-9-octadecenoiate labeled as prepared compound-2.

26. A process for enhancing the properties of solubility, bioavailability, bioabsorbability of atorvastatin wherein one mole of atorvastatin (S)-2,6-diaminohexanoic acid is treated with one mole of pre prepared compound-2 as claimed in claim 25.

27. A process for enhancing the properties of solubility, bioavailability, bioabsorbability of rosuvastatin wherein one mole of rosuvastatin (S)-2,6-diaminohexanoic acid is treated with 2 moles of pre prepared compound-2 as claimed in claim 25.

28. A process as claimed in claim 21 wherein the solubility of the modified form of known pharmaceutical compound(s) is cent percent oil phase and does not separate or precipitate out at a pH from 1 to 8 when dissolved in a aqueous solution with the aid of a known surfactant.

29. A process for enhancing the properties of solubility, bioavailability and bio absorbability of sitosterol wherein one mole of sitosterol is reacted with one mole of mono acylchloride of beta hydroxytricarballyllic acid ((bis 2-aminoethanol di (z)-9-octadecenoiate)).

30. A process for enhancing the properties of solubility, bioavailability and bioabsorbability of torcetrapib wherein one mole of Des N propionyl form of Torcetrapib is reacted with one mole of mono acylchloride of beta hydroxytricarballyllic acid ((bis 2-aminoethanol di (z)-9-octadecenoiate)).

31. A process to obtain the product of claim 1 wherein when:
a. the pharmaceutical or drug having carboxylic acid is treated with amino acid having at least one additional amino group for example with (S)-2,6-diaminohexanoic acid, to form an amide; treating the said amide with hydroxy poly acid such as beta hydroxytricarballyllic acid to form an ester which is further treated with hydroxylamine like 2-aminoethanol to form an ester which is finally treated with long chain fatty acids to get the final product, the process in which the reactants need not be stoichiometrically equivalents; or
b. the pharmaceutical or drug having amino group is made to react with a polyhydroxy polyacid or with a polyacid or with an amino acid such as glutamic acid to form an amide and the remaining procedure is as same as in step (a) to get the final product; or
c. the pharmaceutical or drug having hydroxyl or thiol groups is treated with a poly acid or with a poly hydroxyl poly acid to get an ester, further treated with 2-aminoethanol and followed by the addition of long chain fatty acids to get the final product of the desired properties.

32. A process of achieving the desired blood level of the active drug or pharmaceutical at an administration of lower dosage level of the active drug or pharmaceutical wherein the lower dosage comprises a suitably modified drug as claimed in claim 1.

33. A process of reducing the toxicity of an active drug by administering the modified drug or pharmaceutical as claimed in claim 1 which contain lower dosage of the active drug or pharmaceutical.
